(19) United States
(12) Patent Application Publication
(10) Pub. No.: US 2004/0265377 A1
(43) Pub. Date: Dec. 30, 2004

(54) Solid dispersing vaccine composition for oral delivery

(76) Inventor: Harry Seager, Corsham (GB)

Correspondence Address: Andrew G. Rozycki, Cardinal Health, Inc., 7000 Cardinal Place, Dublin, OH, 43017, US

(21) Appl. No.: 10/835,680

(22) Filed: Apr. 30, 2004

Related U.S. Application Data
(63) Continuation-in-part of application No. 10/136,000, filed on Apr. 29, 2002, now abandoned.
Application No. 10/136,000 is a continuation-in-part of application No. 09/558,560, filed on Apr. 26, 2000, now abandoned.

(30) Foreign Application Priority Data
Oct. 27, 1998 (WO) PCT/GB98/03209
Oct. 27, 1997 (GB) 9722682.3

(51) Int. Cl. A61K 39/12; A61K 9/20

(52) U.S. Cl. 424/464; 424/204.1

(57) ABSTRACT

The invention disclosed herein relate to an oral vaccine in which the vaccine composition and adjuvant(s) are carried on a solid fast-dispersing dosage form. The vaccines are targeted toward mucosal tissue and the adjuvant serves to ensure sufficient residence time for the vaccine composition on the mucosal tissue to facilitate its absorption thereby. The fast-dispersing oral solid vaccine dosage form of the invention is particularly useful to administer the vaccine to patients that have difficulty swallowing medications. In one embodiment, the invention provides a fast disintegrating oral solid vaccine dosage form comprising: an immunogenic amount of an antigenic preparation, the antigenic preparation comprising a microsphere-antigen complex; an adjuvant, wherein the adjuvant enhances the absorption of the antigen or potentiates the immunogenic response; a mucoadhesive substance; and a low density dosage form matrix.

### RELATED APPLICATION DATA

This application is a continuation-in-part of U.S. patent application Ser. No. 10/136,000, now pending, which is a continuation-in-part of U.S. patent application Ser. No. 09/558,560, now abandoned, which is based on International Patent Application No. PCT/GB98/03209 (WO 99/21579) filed on Oct. 27, 1998 which is based on Great Britain Application GB 9722682.3 filed on Oct. 27, 1997.

### FIELD OF THE INVENTION

The invention related to the field of vaccines. In particular, the invention pertains to the field of fast-dissolving oral vaccines in solid dosage forms.

### BACKGROUND OF THE INVENTION

A large variety of dosage forms for oral ingestion are known and readily available in the medical field. Such dosage forms are used for the controlled delivery of medicaments to different parts of the body, the requisite control being achieved by the rate at which the carrier for the medicament breaks down and releases it. Thus, fast dispersing carriers are used for such products in which the medicament is to be quickly released. Slower dispersing carriers and carriers resistant to digestion by gastro-intestinal tract glands can be used where it is intended that release of the medicament is to be delayed, for example, until the product has reached the stomach or lower intestine.

Vaccines, which are important in prophylaxis against disease, exert their effects by provoking an immune response, the effect of which is to prevent infection by the challenging organism, or the onset of the disease process which would otherwise occur when the antigen against which the immune response has been provoked again challenges a sensitive tissue.

Most existing vaccines are delivered by injection, which is traumatic, inconvenient, expensive and may fail to induce an appropriate immunogenic response in the mucosal tissues. Eighty percent of infections affect, or start, in the mucosal surfaces. Active immunization against these infective agents can depend on the successful induction of a mucosal immune response. Successful mucosal vaccines can both protect the secretory surfaces, i.e., mucosal immunity, and also induce systemic immunity by induction of circulatory antibodies. Mucosal vaccines are also easier to administer to patients, and are less expensive to manufacture than conventional vaccines. Delivery by injection does not, of course, directly target the mucosal surfaces or afford the advantages associated with oral vaccines.

The induction of mucosal immunity is evidenced by the appearance of immunoglobulin A antibodies (IgA) in the mucous overlying the mucosa. Successful local stimulation of the mucosal membrane system produces a barrier against a specific pathogen, but this adaptive immunity also confers protection to mucous membranes at other sites in the body. Potentially, oral vaccines can be used to induce immunity against oral, respiratory, genital and ocular pathogens. This ability to generate immunity at sites in the body away from the point of original antigenic stimulation has led to the concept of a common mucosal immune system. There are further indications that stimulation of the mucosal immune system can induce protective circulatory antibodies in the systemic immune system, particularly IgG antibodies.

Vaccines delivered orally can stimulate nasal-associated lymphoid tissue in the mouth and nasal pharyngeal area, the lymph nodes, tonsils and adenoids, and gut-associated lymphoid tissue in the Peyer's patches in the small intestine. FIG. 1 appended hereto illustrates the location of these tissues.

Vaccines incorporate antigens which can be peptides, proteins or whole or partial fragments or extracts of bacterial or viral cells, often attenuated to remove toxic components. In order for vaccines to produce the desired protective effective, systemic exposure to the antigen must be sufficient to provoke an immune response in the recipient. A primary problem in vaccination procedures is ensuring that these antigens or antigenic compounds reach the appropriate site in sufficient quantities to provoke the requisite immune response. There are two aspects of the immune system which can provide the requisite immune response when stimulated by an antigen in a vaccine system: the systemic immune system and the mucosal immune system.

The mucosal immune system consists of areas of lymphoid tissue located in the gastrointestinal tract, the respiratory tract, the genitourinary tract, and the membranes surrounding sensory organs. Such localized areas of lymphoid tissue, when activated by an absorbed antigen, secrete IgA, which exerts an important function in mucosal immunity. Secretory IgA molecules resist proteolysis and mediate antibody-dependent T cell mediated cytotoxicity; inherent microbial adherence, colonization and penetration, as well as food antigen uptake. Stimulation of mucosal tissue can also result in secretion of circulatory IgG antibodies and in turn, IgM and IgE antibodies.

The principal function of the cells forming the lymphoidal tissue is to prevent absorption of pathogens and toxins or to inactivate these pathogens and toxins upon absorption to mucosal tissue. In general, considerably higher doses of antigens are required for mucosalimmunization, especially when intended for the oral route. This is due to the existence of effective mechanical and chemical barriers, and the degradation and digestion of antigens by enzymes and acids. Additionally, there is a rapid clearance of material form the upper respiratory and digestive tracts to the stomach by mucociliary, peristatic and secretory processes.

Difficulty has been encountered in preparing oral solid dosage forms to deliver vaccines through the mucosal route while at the same time preserving ease of administration and patient comfort. Certain patients that have difficulty swallowing are typically poor candidates for solid oral vaccines with increased physical residency in the oral cavity of the dosage form.

There exists a need in the pharmaceutical field for improved oral vaccine dosage forms that effectively deliver immunogenic quantities of antigenic preparations and resist chemical and mechanical barriers to antigenic absorption. There further exists a need for solid oral dosage forms that can induce the immune response effectively as a vaccine while being easy to manufacture and easy and comfortable to administer.

### SUMMARY OF THE INVENTION

The present invention is directed at the use of oral dosage forms of the kind described above to carry vaccines to sites in the human or animal body where they can be best absorbed in a manner which promotes an immune response. It has been discovered that the localized lymphoid tissue associated with effective mucosal vaccine administration can be very effectively targeted by antigens carried on a rapidly disintegrating, water-dispersible solid matrix placed on the tongue. These localized areas of lymphoid tissue, when activated by an absorbed antigen, secrete IgA, which exerts an important function in mucosal immunity. The invention is particularly useful in administering oral vaccines to patients that have difficulty swallowing or otherwise experience discomfort with conventional solid, non-dissolving tablets.

The invention provides a fast-dissolving oral solid vaccine dosage form comprising an immunogenic amount of an antigenic preparation and a low density matrix for oral administration and mucosal absorption. Following placement in the oral cavity and disintegration, the components of the dosage form rapidly coat the mucosal tissues of, and are retained in contact with, the buccopharyngeal region including the mucosal associated lymphoid tissue. Thus, the antigenic components are brought into contact with tissues capable of absorption of the antigen. The dosage form of the invention further comprises adjuvants which enhance the absorption of the vaccine or to potentiate the immunogenic response upon absorption.

Once placed in the oral cavity and in contact with saliva, the fast-dissolving solid oral vaccine dosage forms of the invention can disintegrate preferably within 1 to 60 seconds, more preferably 1 to 30 seconds, especially preferred within 1 to 10 seconds and particularly 2 to 8 seconds. Normally, the disintegration time is less than 60 seconds following the disintegration method specified in United States Pharmacopoeia No. 23, 1995, in water at 37° C. Longer disintegration times are possible if bioadhesive polymers are used in the dosage form composition to extend the residence time of the antigen at the mucosal tissue. Typically, disintegration o the dosage form occurs within a one minute time period.

### BRIEF DESCRIPTION OF THE DRAWINGS

The invention is further illustrated by the following figures, none of which are to be construed as limiting the embodiments of the invention.

FIG. 1 is a schematic diagram of the human body showing the various components of the central (primary) lymphoid system.

FIG. 2 is block diagram showing the geometric mean titre values of total IgA antibodies in saliva samples after administration of TT (Tetanus toxoid) in the various formulations as described in Table 1.

FIG. 3 is a block diagram showing the geometric mean titre values of TT specific IgA antibodies in saliva samples after administration of TT (Tetanus toxoid) in the various formulations as described in Table 1.

### DETAILED DESCRIPTION OF THE INVENTION

As used herein, the phrase “fast dissolving”, “fast dispersing”, and “rapidly disintegrating” when referring to the dosage form of the invention is meant to refer to the capability of the solid dosage form to disintegrate in less than 60 seconds (one minute) of placement in the oral cavity and contact with saliva.

In general, fast dissolving or rapidly dispersing orally administered solid dosage forms can be taken without water and disperse in very small volumes of saliva. This increases the coating of mucosal tissues containing the tonsillar associated lymphoid tissue and increases the residence time of antigens with these tissues. Some fast dispersing solid dosage forms are inherently mucoadhesive. Nevertheless, residence time in contact with the target tissue can be further enhanced by the addition of a mucoadhesive in the dosage form.

The rapid dissolving dosage form promotes delivery of the vaccine to the target site, and the mucoadhesive system can be designed to maintain the vaccine in contact with the target mucosal lymphoid tissues in the mouth and pharynx, and to increase the residence time of the vaccine element at these potential surfaces for absorption. As a product for oral ingestion, from which the vaccine is quickly released once the product is taken, high concentrations of vaccine can thus be quickly delivered to the desired target sites.

Mucoadhesives that can be used in the invention increase the residency of the antigen in contact with the mucosal tissue in the oral cavity and that maintain their adhesive properties following the solid dosage form state. Suitable mucoadhesives that can be used in the invention include, but are not limited to, those described in European Patent Application No. 92109080.9 and include: polyacrylic polymers such as carbomer and carbomer derivatives (e.g., Polycarbophyl™, Carbopol™, and the like); cellulose derivatives such as hydroxypropylmethylcellulose (HPMC), hydroxyethylcellulose (HEC), hydroxypropylcellulose (HPC) and sodium carboxymethylcellulose (NaCPC); and natural polymers such as gelatin, sodium alginate, and pectin. Suitable commercial sources for representative mucoadhesive (bioadhesive) polymers include, but are not limited to, Carbopol™ acrylic copolymer (available from BF Goodrich Chemical Co., Cleveland, Ohio); hydroxypropylmethylcellulose (HPMC) (available from Dow Chemical, Midland, Mich.); HEC (Natrosol) (available from Hercules Inc., Wilmington, Del.); HPC (Kluoel™) (available from Dow Chemical Co., Midland, Mich.); MaCMC (available from Hercules, Inc., Wilmington, Del.); gelatin (available from Deamo Chemical Corp., Elmford, N.Y.); Sodium Alginate (available from Edward Mandell Co., Inc., Carmel, N.Y.); pectin (available from BDH Chemicals Ltd., Poole, Dorset, UK); Polycarbophil™ (available from BF Goodrich Chemical Co., Cleveland, Ohio).

Adjuvants can be used to enhance absorption of the antigen at the target lymphoid tissue and/or to potentiate the immune response resulting from this absorption and stimulation. A variety of such adjuvants can be used with the invention. Suitable adjuvants include, but are not limited to, the following: aluminum salts, non-toxic bacterial fragments, cholera toxin (and detoxified fractions thereof), chitosan, homologous heat-labile of E. coli (and detoxified fractions thereof), lactide/glycolide homo ± and copolymers (PLA/GA), polyanhydride e.g. trimellitylimido-L-tyrosine, DEAE-dextran, saponins complexed to membrane protein antigens (immune stimulating complexes—ISCOMS), bacterial products such as lipopolysaccharide (LPS) and muramyl dipeptide, (MDP), liposomes, cochleates, proteinoids, cytokines (interleukins, interferons), genetically engineered live microbial vectors, non-infectious pertussis mutant toxin, neurimidase/galactose oxidase, and attenuated bacterial and viral toxins derived from mutant strains.

In a preferred embodiment of the invention, the fast dissolving, oral solid vaccine dosage form can include microspheres which can be biodegradable. The microsphere material itself can function as an adjuvant, or can be used in conjunction with other adjuvants. The antigenic preparation can be absorbed or incorporated onto or into microspheres, thereby forming a microsphere-antigenic complex. Thus, the antigenic preparation is available for absorption into the lymphoid tissue effectively as soon as the tissue contacts the microsphere-antigen preparation complex.

Suitable microspheres materials that can be used with the invention include biodegradable polymeric materials. Particularly suitable are hydrophobic materials such as poly(lactic acid) and poly(lactide-co-glycide) polymers, and latex copolymers. These polymeric materials also confer resistance to enzymatic and hydrolytic digestion until their absorption into lymphoid tissue, where the liberated antigen can exert its immunogenic effect. Preferred polymeric materials are hydrophobic materials which enhance absorption into the target tissues.

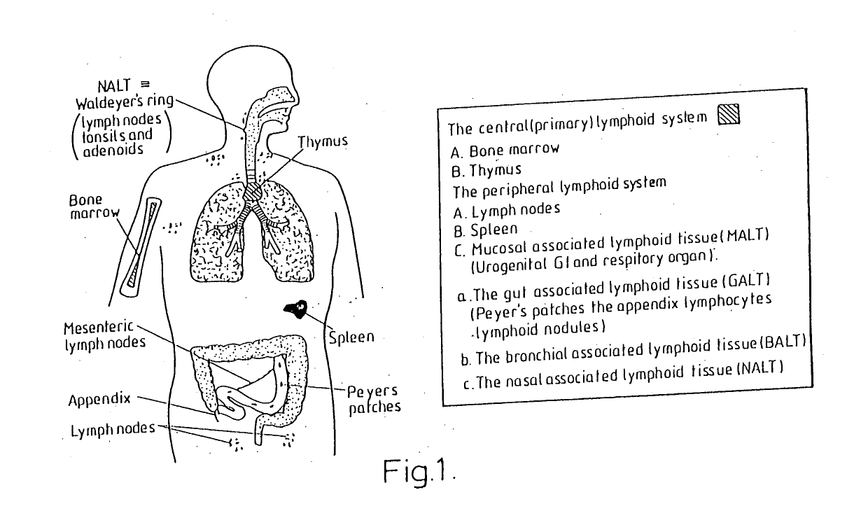

Fast dispersing oral solid dosage forms are known to rapidly disperse and coat the mucosal surfaces in the mouth and pharynx, where the mucosal associated lymphoid tissues are localized. In this respect, reference is directed to a paper by Wilson et al, International Journal of Pharmaceutics, 40 (1997), pages 119-123, the text of which is incorporated herein by reference. FIG. 1 in that paper shows the results of a gamma scintigraphic study. Dosage forms which dissolve rapidly in saliva, with out the aid of water, have also been demonstrated to increase the time in which the rapidly dispersed contents are in contact with the target lymphoid tissue within the buccopharyngeal area and increase the time taken to reach the stomach, when compared to conventional tablets and capsules. Further reference is directed to Wilson et al., International Journal of Pharmaceutics, 46 (1998) pages 241-246); see particularly FIG. 1, incorporated herein by reference. Accordingly, fast-dispersing oral solid dosage forms improve the targeting of vaccines to susceptible lymphoid tissues in the mouth and the pharynx. Consequently, the concentration of vaccine making contact with these tissues increases. Fast-dispersing dosage forms increase the contact time of vaccines with the susceptible lymphoid tissue in the buccopharyngeal area. Furthermore, where antigens are also protected from digestion in the stomach and intestines by ingredients of a dosage form, rapidly dispersed antigenic materials will further target the lymphoid tissue in the Peyer's patches in the small intestine in addition to the oral and laryngeal tissue sites.

Examples of Fast Disintegrating Dosage Forms

One example of a fast-dispersing dosage form is described in U.S. Pat. No. 4,855,326 in which a melt spinnable carrier agent, such as sugar, is combined with an active ingredient and the resulting mixture spun into a “candy-floss” preparation. The spun “candy-floss” product is then compressed into a rapidly dispersing, highly porous solid dosage form.

U.S. Pat. No. 5,120,549 describes a fast-dispersing matrix system which is prepared by first solidifying a matrix-forming system dispersed in a first solvent, and subsequently contacting the solidified matrix with a second solvent that is substantially miscible with the first solvent at a temperature lower than the solidification point of the first solvent. As the matrix-forming elements and active ingredient are substantially insoluble in the second solvent, the first solvent is substantially removed resulting in a fast-dispersing matrix.

U.S. Pat. No. 5,079,018 describes a fast-dispersing dosage form which comprises a porous skeletal structure of a water soluble, hydratable gel or foam forming material that has been hydrated with water, rigidified in the hydrated state with a rigidifying agent and dehydrated with a liquid organic solvent at a temperature of about 0° C. or below to leave spaces in place of hydration liquid.

Published International Application No. WO 93/12769 (PCT/JP93/01631) describes fast-dispersing dosage forms of very low density formed by gelling, with agar, aqueous systems containing the matrix—forming elements and active ingredient, and then removing water by forced air or vacuum drying.

U.S. Pat. No. 5,298,261 describes a fast-dispersing dosage forms which comprise a partially collapsed matrix network that has been vacuum-dried above the collapse temperature of the matrix. However, the matrix is preferably at least partially dried below the equilibrium freezing point of the matrix.

Published International Application No. WO 91/04757 (PCT/US90/05206) discloses fast-dispersing dosage forms which contain an effervescent disintegration agent designed to effervesce on contact with saliva to provide rapid disintegration of the dosage form and dispersion of the active ingredient in the oral cavity.

U.S. Pat. No. 5,595,761 discloses a particulate support matrix for use in making a rapidly dissolving tablet, comprising;

a first polypeptide component having a net charge when in solution, e.g. non-hydrolyzed gelatin;

a second polypeptide component having a net charge of the same sign as the net charge of the first polypeptide component when in solution, e.g. hydrolyzed gelatin; and

a bulking agent, and wherein the first polypeptide component and the second polypeptide component together comprise about 2% to 20% by weight of the particulate support matrix and wherein the bulking agent comprises about 60% to 96% by weight of the particulate support matrix; and

wherein the second polypeptide component has a solubility in aqueous solution greater than that of the first polypeptide component and wherein the mass: mass ratio of the first polypeptide component to the second polypeptide component is from about 1:½ to about 1:14; and

wherein when the support matrix is introduced into an aqueous environment, the support matrix is disintegrable within less than about 20 seconds.

U.S. Pat. No. 5,576,014 describes a fast-dispersing dosage form which dissolves intrabuccally and which comprises compressed moldings formed from granules comprising a saccharide having low moldability which has been granulated with a saccharide having high moldability. The resulting compressed moldings show quick disintegration in the buccal cavity.

European Patent No. 690,747 B1 describes particles comprising an excipient forming a matrix and at least one active ingredient uniformly distributed in the mass of the matrix which are prepared by a process comprising the steps of preparing an homogeneous pasty mixture with a viscosity below 1 Pa.s, measured at room temperature (15-20° C.), from at least one active ingredient, a physiologically acceptable hydrophilic excipient and water; extruding the resulting homogenous mixture and cutting the extrudate to give moist particles; freezing the resulting particles as they fall under gravity through a stream of inert gas at a temperature below 0°; and drying the particles by freeze drying.

Australian Patent No. 666,666 describes a rapidly disintegratable multiparticulate tablet having a mixture of excipients in which the active substance is present in the form of coated microcrystals or optionally coated microgranules. Such tablets disintegrate in the mouth in an extremely short time, typically less than 60 seconds.

U.S. Pat. No. 5,382,437 discloses a porous carrier material having sufficient rigidity for carrying and administering an active material which is capable of rapid dissolution by saliva and which is formed by freezing a liquified ammonia solution comprising liquid ammonia, a liquid ammonia-soluble gel or foam material and a rigidifying agent for the gel or foam material selected from the group consisting of a monosaccharide, a polysaccharide and combinations thereof, and deammoniating the frozen material thus formed by causing material transfer of ammonia from the frozen state to the gas state thereby leaving spaces in the carrier material in place of the frozen ammonia

Published International Application No. WO 93/13758 (PCT/US92/07497) describes tablets of increased physical strength which disintegrate in the mouth in less than 10 second and which are prepared by combining and compressing a meltable binder, excipients and a pharmaceutically active agent into a tablet, melting the binder in the tablet and then solidifying the binder.

U.S. Pat. Nos. 3,285,026 and 4,134,943 also describe fast-dispersing porous tablets and a method for increasing their physical strength by first compressing the tablet and then volatilizing a readily volatilizable solid adjuvant incorporated in the tablet to attain the desired porosity.

European Patent Application No. 601,965 describes a shearform matrix material which can be used, inter alia, to deliver a pharmaceutically active agent. The shearform matrix is formed by increasing the temperature of a feedstock which includes a solid non-solubilized carrier material to the point where it will undergo internal flow with the application of a fluid shear force, ejecting a stream of the heated feedstock thus formed under pressure from an orifice and then subjecting the feedstock to disruptive fluid shear force which separates the flow of feedstock into multiple parts and transforms the morphology of the feedstock.

U.S. Pat. No. 5,683,720 discloses discrete particles containing a pharmaceutically active agent which can be fast-dispersing and are formed by subjecting a solid, organic feedstock to liquiflash conditions whereby the feedstock is transformed instantaneously from solid to liquiform to solid, liquiform being a transient condition in which the feedstock has substantially unimpeded internal flow. Shear force is then imparted to the liquiform feedstock in an amount sufficient to separate tiny masses of feedstock which then solidify as discrete particles.

U.S. Pat. No. 5,576,014 discloses fast-dispersing dosage forms in the form of intrabuccally dissolving compressed moldings comprising a saccharide having low moldability which has been granulated with a saccharide having high moldability.

Published International Application No. WO 95/34293 describes the preparation of fast-dispersing dosage forms comprising a three-dimensional crystalline-based porous network bound together to form a stable structure which is formed by mixing uncured shearform matrix and an additive, molding the dosage form and curing the shearform matrix.

European Patent Application No. 737,473 discloses fast-dispersing dosage forms which are effervescent. Each such dosage form comprises a mixture of at least one water or saliva activated effervescent agent and a plurality of microcapsules containing the active ingredient.

U.S. Pat. No. 5,587,180 describes fast-dispersing dosage forms which include an active ingredient and a particulate support matrix comprising a first polymeric component which may be a polypeptide such as a non-hydrolyzed gelatin, a second polymeric component which may be a different polypeptide such as a hydrolyzed gelatin and a bulking agent. Generally, the dosage forms are prepared by mixing the particulate support matrix with the active ingredient and any other additives and then forming the mixture into tablets by compression.

European Patent Application No. 0627,218 disclose a fast-dispersing dosage form which comprises a tablet comprising a sugar alcohol or the like as a principal ingredient which is prepared by the wet granulation method in which a kneaded mixture of the sugar alcohol or the like with a drug is compression molded before drying.

Published International Application No. WO 94/14422 describes a process for drying frozen discrete units in which the solvent is removed under conditions whereby the solvent is evaporated from the solid through the liquid phase to a gas, rather than subliming from a solid to a gas as in lyophilization. This is achieved by vacuum drying at a temperature below the equilibrium freezing point of the composition at which point the solvent (such as water) changes phase.

Fast dispersing dosage forms that can be used in accordance with the invention include the types of solid dosage forms described herein above in the preceding paragraphs. Particularly preferred fast disintegrating dosage forms for use with the invention is that described in U.K. Patent No. 1,548,022, which is directed to a solid fast-dispersing solid oral dosage form comprising a network of the active ingredient and a water-soluble or water-dispersible carrier which is inert towards the active ingredient, the network having been obtained by subliming solvent from a composition comprising the active ingredient and a solution of the carrier in a solvent.

In the case of the preferred type of fast-dispersing dosage form described above, the composition will preferably contain, in addition to the antigenic active ingredient, matrix forming agents and secondary components. Matrix forming agents suitable for use in the present invention include materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

Other matrix forming agents suitable for use in the present invention include sugars such as mannitol, dextrose, lactose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine.

One or more matrix forming agents may be incorporated into the solution or suspension prior to solidification. The matrix forming agent may be present in addition to a surfactant or to the exclusion of a surfactant. In addition to forming the matrix, the matrix forming agent may aid in maintaining the dispersion of any active ingredient with the solution or suspension. This is especially helpful in the case of active agents that are not sufficiently soluble in water and must, therefore, be suspended rather than dissolved.

Secondary components such as preservatives, antioxidants, surfactants, viscosity enhancers, coloring agents, flavoring agents, pH modifiers, sweeteners or taste-masking agents may also be incorporated into the composition. Suitable coloring agents include red, black and yellow iron oxides and FD & C dyes such as FD & C blue No. 2 and FD & C red No. 40 available from Ellis & Everard. Suitable flavoring agents include mint, raspberry, liquorice, orange, lemon, grapefruit, caramel, vanilla, cherry and grape flavors and combinations of these. Suitable pH modifiers include citric acid, tartaric acid, phosphoric acid, hydrochloric acid and maleic acid. Suitable sweeteners include aspartame, acesulfame K and thaumatic. Suitable taste-masking agents include sodium bicarbonate, ion-exchange resins, cyclodextrin inclusion compounds, adsorbates or microencapsulated actives.

The fast disintegrating solid oral vaccine dosage form of the present invention might, for example, be used for the delivery of vaccines designed to prevent or reduce the symptoms of diseases of which the following is a representative but not exclusive list:

Influenza, Tuberculosis, Meningitis, Hepatitis, Whooping Cough, Polio, Tetanus, Diphtheria, Malaria, Cholera, Herpes, Typhoid, HIV, AIDS, Measles, Lyme disease, Travellers' Diarrhea, Hepatitis A, B and C, Otitis Media, Dengue Fever, Rabies, Parainfluenza, Rubella, Yellow Fever, Dysentery, Legionnaires Disease, Toxoplasmosis, Q-Fever, Haemorrhagic Fever, Argentina Haemorrhagic Fever, Caries, Chagas Disease, Urinary Tract Infection caused by E.coli, Pneumoccoccal Disease, Mumps, and Chikungunya.

The dosage form of the invention can further be used to prevent or reduce the symptoms of other disease syndromes of which the following is a representative but not exclusive list of causitive organisms:

Vibrio species, Salmonella species, Bordetella species, Haemophilus species, Toxoplasmosis gondii, Cytomegalovirus, Chlamydia species, Streptococcal species, Norwalk Virus, Escherischia coli, Helicobacter pylori, Rotavirus, Neisseria gonorrhae, Neisseria meningiditis, Adenovirus, Epstein Barr virus, Japanese Encephalitis Virus, Pneumocystis carini, Herpes simplex, Clostridia species, Respiratory Syncytial Virus, lebsielia species, Shigella species, Pseudomonas aeruginosa, Parvovirus, Camylobacter species, Rickettsia species, Varicella zoster, Yersinia species, Ross River Virus, J.C. Virus, Rhodococcus equi, Moraxella catarrhalis, Borrelia burgdorferi and Pasteurella haemolytica.

The fast dissolving oral solid vaccine dosage form of the invention can also be used with vaccines directed to non-infections immuno-modulated disease conditions such as topical and systematic allergic conditions such as Hayfever, Asthma, Rheumatoid Arthritis and Carcinomas.

Veterinary applications of the invention are also contemplated. Vaccines for veterinary use include those directed to Coccidiosis, Newcastle Disease, Enzootic pneumonia, Feline Leukemia, Atrophic rhinitis, Erysipelas, Foot and Mouth disease, Swine, pneumonia, and other disease conditions and other infections and auto-immune disease conditions affecting companion and farm animals.

### EXAMPLE 1

### Comparative In Vivo Immunogenicity Data of Fast Dispersing Oral Solid Vaccine Dosage Forms using Tetanus Toxoid (TT) and Other Administration Routes

In a preliminary test, the immunogenicity of tetanus toxoid (TT) in twenty-five rabbits was studied following oral delivery in fast dispersing dosage forms (FDDF) of the kind described in British Patent No. 1,548,022. For comparative reference, similar tests were conducted using oral administration of TT in solution, and intramuscular administration by injection of TT adsorbed to aluminum hydroxide. The administered formulations are set out in Table 1 in which the TT concentration is suppressed as the concentraion of TT protein. The adjuvants used in Formulations 1 to 3, PLSP and chitosan, are discussed in more detail in published International Patent Application Nos. WO97/02810 and WO90/09780. A summary of the dose groups is given in Table 2. Oral administration of Formulations Nos. 1 to 3 was by placement of the FDDF unit at the rear of the tongue after spraying the oral cavity with 0.12 ml. of UHP water after which the oral cavity was against sprayed with 0.06 ml. of UHP water. Formulation 4 was delivered in a dose of 0.5 ml by syringe delivered to the rear of the tongue. Formulation 5 was delivered by injection of a 0.2 ml dose to the quadriceps (front thigh) to muscles of the left hind limb. Prior to each dose administration, and at termination blood and saliva samples were collected. The dosing and sampling schedule is set out in Table 3.

TABLE 1
Administered Formulations

| Formulation | Type of | | Route of |
|---|---|---|---|
| No. | Formulation | Outline Composition | Administration |
| 1 | FDDF unit | 0.4 mg TT/PLSP/ | Oral |
| | | Gelatin/Mannitol | |
| 2 | FDDF unit | 0.4 mm TT/Chitosan/ | Oral |
| | | Gelatin/Mannitol | |
| 3 | FDDF unit | 0.4 mg TT/Chitosan/ | Oral |
| | | PLSP/Gelatin/Mannitol | |
| 4 | Solution | 0.8 mg/ml TT in water | Oral |
| 5 | Suspension | 0.4 mg/ml TT/alum | IM |

TABLE 2
Dose Group Summary

| | Type of | | | | | | | |
|---|---|---|---|---|---|---|---|---|
| Formulation | Formulation | Rabbit | (mg/rabbit) | | | | | |
| or Group No. | (Route) | No. | TT | Chitosan | PLSP | Gelatin | Mannitol | Alum |
| 1 | FDDF unit | 1-5 | 0.4 | — | 10 | 15 | 15 | — |
| | (oral) | | | | | | | |
| 2 | FDDF unit | 6-10 | 0.4 | 5 | — | 5 | 5 | — |
| | (oral) | | | | | | | |
| 3 | FDDF unit | 11-15 | 0.4 | 5 | 10 | 5 | 5 | — |
| | (oral) | | | | | | | |
| 4 | Solution | 16-20 | 0.4 | — | — | — | — | — |
| | (oral) | | | | | | | |
| 5 | Suspension | 21-25 | 0.08 | — | — | — | — | 4.8 |
| | (IM) | | | | | | | |

TABLE 3
Dosing and Sampling Schedule

| | Study | |
|---|---|---|
| Study Date | Day | Procedure |
| Jul. 31, 1998 | 1 | Collect pre-dose saliva samples from rabbits 1-25 |
| | | Dose rabbits 1-25 with appropriate formulations |
| | | (refer to Table 2) |
| Aug. 20, 1998 | 21 | Collect pre-dose saliva samples from rabbits 1-25 |
| | | Dose rabbits 1-25 with appropriate formulation |
| | | (refer to Table 2) |
| Sep. 10, 1998 | 42 | Collect pre-dose saliva samples from rabbits 1-25 |
| | | Dose rabbits 1-25 with appropriate formulation |
| | | (refer to Table 2) |
| Sep. 24, 1998 | 56 | Collect terminal saliva samples from rabbits 1-25 |

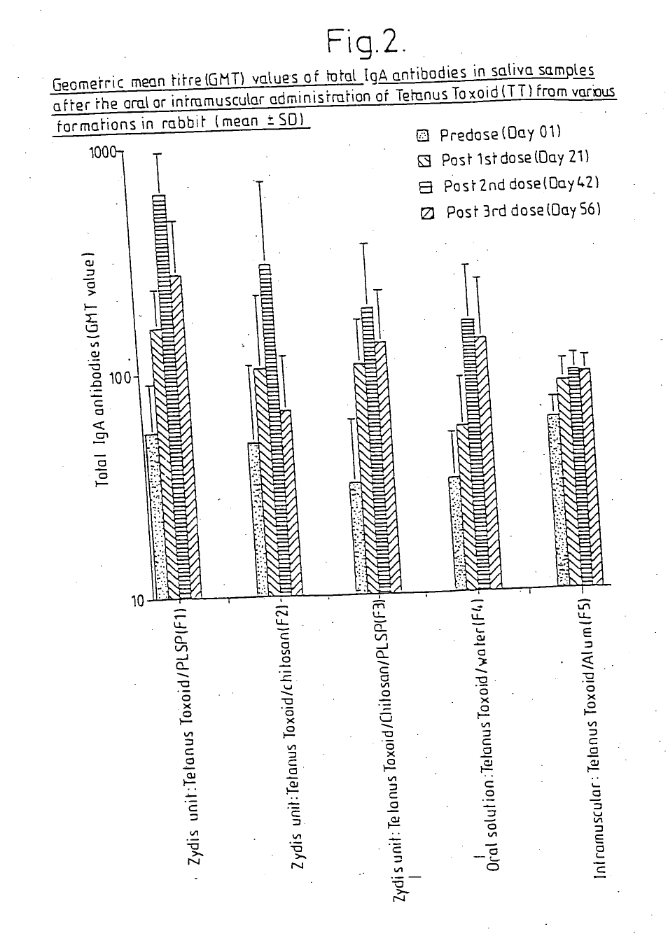

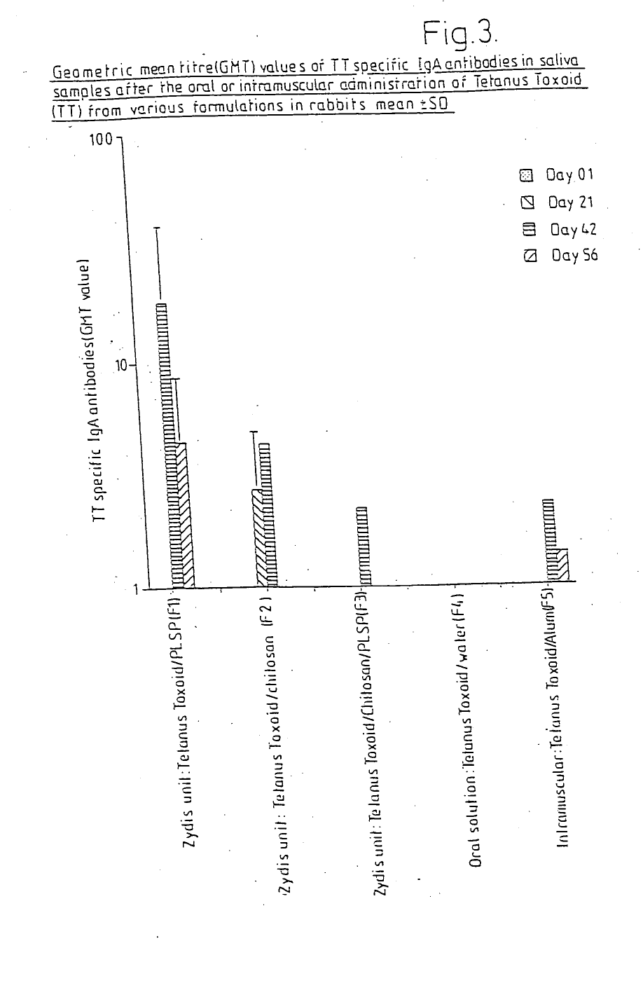

FIG. 2 is a block diagram showing the geometric mean titre values of total IgA antibodies in saliva samples after administration of TT in the various formulations of Table 1 above (mean±SD). As can be seen from the diagram (the ordinate is on a logarithmic scale) the peak IgA values achieved using formulations 1 to 3 are significantly better than those for formulation 5 with formulation 1 providing the best figures by a considerable margin. Similar tests were conducted to monitor TT specific antibodies. The results are illustrated in FIG. 3. Even on a lower logarithmic scale, Formulations 1 and 2 show significant improvement in immune response relative to the intramuscular delivered dosage, Formulations 5.

It should be noted that only saliva samples that exhibited a positive response to the assay text were recorded. This explains the apparent absence of any immune response at some stages for some formulations, and the apparent absence of response at any stage for Formulation 4. The tests showed some response at these stages, but not any there were felt to be statistically significant. Based on the results, the improved immune response exhibited by Formulations 1 and 3 in FIG. 3 demonstrates the potential benefit of administering vaccines with one or more adjuvants in a fast dispersing dosage form, by oral delivery.

The complete disclosure of all patents, patent applications and publications are incorporated herein by reference as if each were individually incorporated by reference. The present invention has been described with reference to various specific and preferred embodiments and techniques. It will be understood by one of ordinary skill, however, that reasonable variations and modifications can be made while remaining within the spirit and scope of the invention defined by the claims below.

What is claimed is:

1. A fast disintegrating oral solid vaccine dosage form comprising:
an immunogenic amount of an antigenic preparation, said antigenic preparation comprising a microsphere-antigen complex;
an adjuvant, wherein said adjuvant enhances the absorption of the antigen or potentiates the immunogenic response;
a mucoadhesive substance; and
a low density dosage form matrix.

2. The oral vaccine dosage form according to claim 1, wherein the dosage form disintegrates within 60 seconds after being placed in the oral cavity.

3. The oral vaccine dosage form according to claim 2, wherein the dosage form disintegrates within 30 seconds of being placed in the oral cavity.

4. The oral vaccine dosage form according to claim 3, wherein the dosage form disintegrates within 10 seconds of being placed in the oral cavity.

5. The oral vaccine dosage form according to claim 1, wherein said micropshere of said microsphere-antigen complex comprises antigen encapsulated in biodegradable polymeric material.

6. The oral vaccine dosage form according to claim 5, wherein said biodegradable polymeric material is selected from poly(lactic acid), poly (lactide-co-glycide), and combinations thereof.

7. The oral vaccine dosage form according to claim 1, wherein said microsphere of said microsphere-antigenic complex comprises latex.

8. The oral vaccine dosage form according to claim 1, wherein the low density matrix is formed by removal of solvent by lyophilization from a frozen suspension.

9. The oral vaccine dosage form according to claim 1, wherein the low density matrix is formed by removal of solvent from a frozen suspension by contact with a second solvent, in which the matrix forming materials are insoluble.

10. The oral vaccine dosage form according to claim 1, wherein low density matrix is formed by compacting finely divided extruded materials.

11. The oral vaccine dosage form according to claim 1 wherein the low density matrix is formed by loosely compacting particles formed by spray-coating, spray drying, spray-chilling, coaservation or fluid-bed drying.

12. The oral vaccine dosage form according to claim 1, wherein the low density matrix is formed by gelling a suspension and then removing solvent by drying.

13. The oral vaccine dosage form according to claim 1, wherein said microsphere-antigen complex comprises an antigenic preparation is absorbed onto polymeric particles.

14. The oral vaccine dosage form according to claim 1, wherein said mucoadhesive increases the residency of the antigen in contact with the mucosal tissue in the oral cavity, and maintains adhesive properties following the solid dosage form state.

15. The oral vaccine dosage form according to claim 14, wherein the mucoadhesive is a polyacrylic polymer.

16. The oral vaccine dosage form according to claim 14, wherein the mucoadhesive is a cellulose derivative.

17. The oral vaccine dosage form according to claim 14, wherein the mucoadhesive is selected from gelatin, sodium alginate, and pectin.

18. A fast disintegrating oral solid vaccine dosage form comprising:
an immunogenic amount of an antigenic preparation comprising tetanus toxoid (TT), said antigenic preparation comprising a microsphere-antigen complex;
an adjuvant, wherein said adjuvant enhances the absorption of the antigen or potentiates the immunogenic response;
a mucoadhesive substance; and
a low density dosage form matrix.

19. The oral vaccine dosage form according to claim 18, wherein the dosage form disintegrates within 60 seconds after being placed in the oral cavity.

20. The oral vaccine dosage form according to claim 19, wherein the dosage form disintegrates within 30 seconds of being placed in the oral cavity.

21. The oral vaccine dosage form according to claim 20, wherein the dosage form disintegrates within 10 seconds of being placed in the oral cavity.

22. The oral vaccine dosage form according to claim 18, wherein said micropshere of said microsphere-antigen complex comprises antigen encapsulated in biodegradable polymeric material.

23. The oral vaccine dosage form according to claim 22, wherein said biodegradable polymeric material is selected from poly(lactic acid), poly (lactide-co-glycide), and combinations thereof.

24. The oral vaccine dosage form according to claim 18 wherein said microsphere of said microsphere-antigenic complex comprises latex.

25. The oral vaccine dosage form according to claim 18, wherein the low density matrix is formed by removal of solvent by lyophilization from a frozen suspension.

26. The oral vaccine dosage form according to claim 18, wherein the low density matrix is formed by removal of solvent from a frozen suspension by contact with a second solvent, in which the matrix forming materials are insoluble.

27. The oral vaccine dosage form according to claim 18, wherein low density matrix is formed by compacting finely divided extruded materials.

28. The oral vaccine dosage form according to claim 18, wherein the low density matrix is formed by loosely compacting particles formed by spray-coating, spray drying, spray-chilling, coaservation or fluid-bed drying.

29. The oral vaccine dosage form according to claim 18, wherein the low density matrix is formed by gelling a suspension and then removing solvent by drying.

30. The oral vaccine dosage form according to claim 18, wherein said microsphere-antigen complex comprises an antigenic preparation is absorbed onto polymeric particles.

31. The oral vaccine dosage form according to claim 18, wherein said mucoadhesive increases the residency of the antigen in contact with the mucosal tissue in the oral cavity, and maintains adhesive properties following the solid dosage form state.

32. The oral vaccine dosage form according to claim 18, wherein the mucoadhesive is a polyacrylic polymer.

33. The oral vaccine dosage form according to claim 18, wherein the mucoadhesive is a cellulose derivative.

34. The oral vaccine dosage form according to claim 18, wherein the mucoadhesive is selected from gelatin, sodium alginate, and pectin.
